(19) United States
(12) Patent Application Publication
(10) Pub. No.: US 2017/0196987 A1
(43) Pub. Date: Jul. 13, 2017

(54) METHODS FOR TREATING MULTIPLE SCLEROSIS

(71) Applicant: MedWell Laboratories Ltd., Nazareth (IL)

(72) Inventor: Taher NASSER, Tur'an (IL)

(21) Appl. No.: 15/313,955

(22) PCT Filed: May 21, 2015
(86) PCT No.: PCT/IL2015/050538

§ 371 Date: Nov. 24, 2016

Related U.S. Application Data
(60) Provisional application No. 62/002,872, filed on May 25, 2014.

(51) Int. Cl. A61K 31/4015; A61K 9/00; A61K 31/202; A61K 31/401; A61K 31/57; A61K 39/395; A61K 38/21; A61K 38/14; A61K 31/573

(52) U.S. Cl. CPC A61K 47/48038; A61K 31/401; A61K 31/202; A61K 47/481; A61K 9/0019

(57) ABSTRACT

This invention is directed to a method for treating or preventing multiple sclerosis (MS), treating multiple sclerosis symptoms or preventing the continual chronic deterioration caused by multiple sclerosis (MS), comprising administering a conjugate of hydroxyproline and docosahexaenoic acid (DHA) according to formula MW-001.

### FIELD OF THE INVENTION

The invention is directed to methods for treating or preventing multiple sclerosis comprising administering a conjugate of docosahexaenoic acid (DHA) and hydroxyproline.

### BACKGROUND OF THE INVENTION

4-hydroxyproline (4-hydroxy 2-pyrrolidine-carboxylic acid, related to herein as hydroxyproline) is a naturally occurring amino acid. 4-hydroxyproline is not considered to be an essential amino acid and further, no pharmacological activity for 4-hydrxyproline is known in the field.

However, certain N-substituted derivatives of hydroxproline containing substituted peptides are known ACE-inhibitors and have become established for their ability to control high blood pressure (for example Captopril; Lisinopril; Enalapril and Moveltipril). Oxaceprol, an additionally N-acyl derivative; possesses antiphlogistic; anti-rheumatic and wound healing activity. Commercial infusions for parenteral nutrition occasionally contain proline as an adjuvant (for example, in combination with other amino acids, carbohydrates and electrolytes).

Certain Omega-3 and Omega-6 fatty acids are considered essential fatty acids, which, although essential for human health, cannot be produced by the human body. Other omega fatty acids are known to be conditionally essential, i.e., essential to the human body under certain conditions. Omega fatty acids can be found in fish, seafood andcertain plants. Also known as polyunsaturated fatty acids (PUFAs), omega fatty acids play a crucial role in brain function, as well as normal growth and development. They have also become popular since they may reduce the risk of heart disease. Research shows that omega fatty acids reduce inflammation and may help lower risk of chronic diseases such as heart disease, cancer, and arthritis. Omega fatty acids are highly concentrated in the brain and appear to be important for cognitive and behavioral function. Symptoms of omega fatty acid deficiency include fatigue, poor memory, dry skin and heart problems.

Multiple Sclerosis (MS) is a chronic disease of the central nervous system (CNS), associated with demyelination and neurodegeneration. MS exhibits many of the hallmarks of an inflammatory autoimmune disorder including breakdown of the blood—brain barrier (BBB); the recruitment of lymphocytes, microglia, and macrophages to lesion sites; the presence of multiple CNS lesions. Multiple etiologies including autoimmunity, infectious agents, environmental triggers and hereditary factors have been proposed. However, there is substantial evidence indicating that dysregulated immune responses, including immune mechanisms directed against myelin proteins, have a role in triggering disease onset.

Typically, MS affects the brain, spinal cord, and optic nerves in the CNS and spares the nerve roots and peripheral nerves in the peripheral nervous system. The interplay between inflammatory and neurodegenerative processes in MS typically results in intermittent neurological disturbance (episodes of acute worsening) followed by the progressive accumulation of disability. During an MS attack, inflammation occurs in areas of the white matter of CNS in patches known as “plaques”. This process is followed by destruction of myelin in the brain and spinal cord, leading to diminished or lost function.

### SUMMARY OF THE INVENTION

In an embodiment of the invention, there is provided a method for treating multiple sclerosis (MS) or treating multiple sclerosis symptoms comprising administering a conjugate of hydroxyproline and docosahexaenoic acid (DHA) according to formula

MW-001:

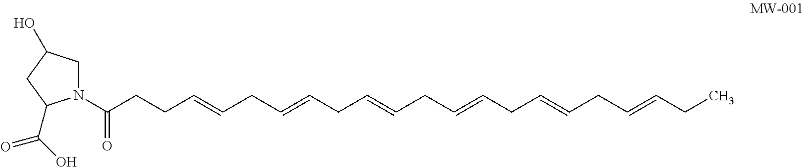 [SMILES: CC/C=C/C/C=C/C/C=C/C/C=C/C/C=C/C/C=C/CCC(=O)N1CC(O)CC1C(=O)O]

In some embodiments of the invention, there is provided a method for preventing the outburst of multiple sclerosis (MS) comprising administering a conjugate of hydroxyproline and docosahexaenoic acid (DHA) according to formula MW-001:

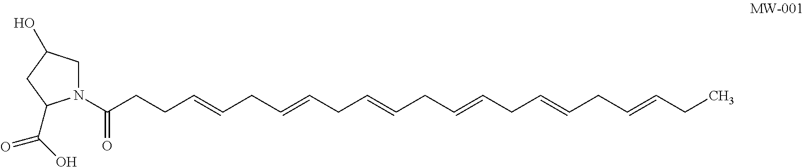 [SMILES: CC/C=C/C/C=C/C/C=C/C/C=C/C/C=C/C/C=C/CCC(=O)N1CC(O)CC1C(=O)O]

In some embodiments, there is provided a method for preventing the continual chronic deterioration caused by multiple sclerosis (MS) comprising administering a conjugate of hydroxyproline and docosahexaenoic acid (DHA) according to formula MW-001:

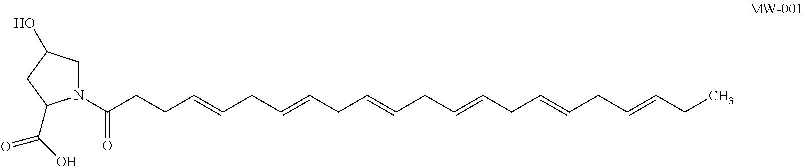 [SMILES: CC/C=C/C/C=C/C/C=C/C/C=C/C/C=C/C/C=C/CCC(=O)N1CC(O)CC1C(=O)O]

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 is a graph presenting the mean clinical score of untreated EAE mice, EAE mice treated with a conjugate of hydroxyproline and docosahexaenoic acid (DHA) i.e. MW-001 (related to herein also as MWL-001) from day 0 and EAE mice treated with the same conjugate from day 10.

FIG. 2 is a graph presenting the mean clinical score of untreated EAE mice, EAE mice treated orally with MW-001 (related to herein also as MWL-001), and EAE mice treated ip with MW-001.

FIG. 3 is a graph presenting the mean clinical score of EAE mice treated with MP, EAE mice treated with MW-001 (related to herein also as MWL-001), and EAE mice treated with MW-001+MP (methylprednisolone).

FIG. 4 is a graph presenting the in vitro effect of MWL-001 on the activation of human lymphocytes.

FIG. 5 (A, B and C) is a histopathological photograph of a spinal cord section stained with H&E of control untreated EAE animal. P=parenchymal infiltrates, M=meningeal infiltrates and D=diffused infiltrates.

FIG. 6 (A and B) is a histopathological photograph of a spinal cord section stained with H&E of EAE animal treated with MWL-001 (i.p.) on day 0.

FIG. 7 (A and B) is a histopathological photograph of a spinal cord section stained with H&E of EAE animal treated with MWL-001 (i.p.) on day 10.

### DESCRIPTION OF THE DETAILED EMBODIMENTS OF THE INVENTION

According to one embodiment of the invention, there is provided a method for treating or preventing the outburst of multiple sclerosis (MS) and/or preventing continual chronic deterioration caused by MS comprising administering a conjugate of hydroxyproline and docosahexaenoic acid (DHA) according to formula MW-001:

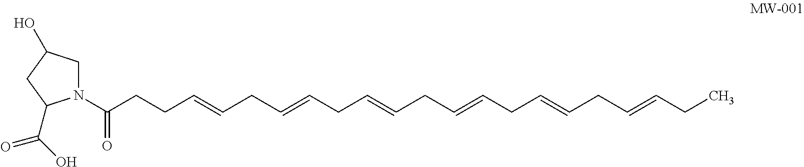 [SMILES: CC/C=C/C/C=C/C/C=C/C/C=C/C/C=C/C/C=C/CCC(=O)N1CC(O)CC1C(=O)O]

The terms “treatment” or “treating” is intended to encompass therapy, preventing relapse, and amelioration of acute symptoms. Note that “treating” refers to either or both of the amelioration of symptoms and the resolution of the underlying condition. In many of the embodiments, the administration of a compound or composition may act not directly on the disease state, but rather on some pernicious symptom, and the improvement of that symptom leads to a general and desirable amelioration of the disease state.

As used herein the phrase “Multiple Sclerosis” refers to the inflammatory, demyelinating disease of the central nervous system (CNS) which is typically characterized by various symptoms of neurological dysfunction. Any type of Multiple Sclerosis (MS) may be treated according to the teachings of the present invention including relapsing-remitting, secondary progressive, primary progressive, progressive relapsing and special cases of MS with non-standard behavior (also referred to as borderline forms of MS), such as for example without limitation, Neuromyelitis optica (NMO), Balo concentric sclerosis, Schilder disease, Marburg multiple sclerosis, acute disseminated encephalomyelitis (ADEM) and autoimmune variants of peripheral neuropathies. The disease may be treated at any stage including, but not limited to when the subject is undergoing an acute attack. According to some embodiments, the disease may be treated chronically for preventing outbreaks and the damage caused therefrom, during flare ups of the disease for reducing and preventing deterioration or both. According to some embodiments, the conjugate is administered before the outbreak of the disease. According to some embodiments, the disease is treated in patients that are resistant and/or not sensitive to conventional treatments, such as treatments with steoroids. According to some embodiments, the symptoms of the disease are treated by the conjugate as detailed herein.

According to some embodiments, the conjugate is administered orally, possibly in the form of tablets, capsules, powders, troches, soft gelatin capsules, syrup, liquid suspension or lozenges. According to some embodiments, the conjugate is administered in the form of a parenteral formulation, such as for subcutaneous intramuscular or intravenous administration. According to further embodiments, the conjugate is administered by any appropriate nasal administration, pulmonary administration, topically or be any appropriate dermatological administration.

According to some embodiments, the conjugate is administered together with any appropriate non-toxic pharmaceutical carrier. The carrier may be a gas, solid or liquid. According to some embodiments, the carrier is selected from saline solution, water, any appropriate emulsion or dispersion or any combination thereof.

According to some embodiments, the conjugate is administered together with any ingredients appropriate for modifying the release of the active conjugate from the formulation. Time delaying agents, such as enteric coated gelatin capsules may be used.

According to some embodiments, the conjugate may be administered together with any additional active ingredients, such as without being limited, Interferon beta 1a, Interferon beta 1b, Glatiramer acetate, mitoxantrone, methyl-prednisolone, prednisone, prednisolone, dexamethasone, adreno-corticotrophic hormone (ACTH), corticotrophin, beta interferons, corticostaeorids, natalizumab (Tysabri®), fingolimod (Gilenya®), glatiramer acetate (Copaxone®), mitoxantrone teriflunomide (Aubagio®) or any appropriate combination thereof.

According to some embodiments, the conjugate may be administered in an amount of from about 1 mg/kg to 1000 mg/kg. According to some embodiments, the conjugate may be administered in an amount of about 1-10 mg/kg. According to some embodiments, the conjugate may be administered in an amount of about 10-50 mg/kg.

According to some embodiments, the conjugate may be administered in an amount of about 50-100 mg/kg. According to some embodiments, the conjugate may be administered in an amount of about 100-200 mg/kg. According to some embodiments, the conjugate may be administered in an amount of about 200-300 mg/kg. According to some embodiments, the conjugate may be administered in an amount of about 300-400 mg/kg. According to some embodiments, the conjugate may be administered in an amount of about 400-500 mg/kg. According to some embodiments, the conjugate may be administered in an amount of about 500-600 mg/kg. According to some embodiments, the conjugate may be administered in an amount of about 600-700 mg/kg. According to some embodiments, the conjugate may be administered in an amount of about 700-800 mg/kg. According to some embodiments, the conjugate may be administered in an amount of about 800-900 mg/kg. According to some embodiments, the conjugate may be administered in an amount of about 900-1000 mg/kg.

According to some embodiments, the conjugate may be administered once a day. According to other embodiments, the conjugate are administered twice a day, three times a day or more.

According to some embodiments, the conjugate is administered chronically. According to some embodiments, the composition is administered for about 10 days or more, 20 days, 30 days, 60 days, 90, 120 days or more.

As defined herein, treating and preventing includes fully healing a condition, partially healing a condition, such that an improvement occurs and preventing, or at least partially preventing, the occurrence of the condition.

The term “preventing” refers to keeping a disease, disorder or condition from occurring in a subject. In some cases the subject may be at risk for developing the disease, but has not yet been diagnosed as having the disease. In some instances, the term “preventing” refers to preventing the next cycle of the disease from occurring.

As used herein the term “about” refers to ±10%.

Various aspects of the invention are described in greater detail in the following Examples, which represent embodiments of this invention, and are by no means to be interpreted as limiting the scope of this invention.

### EXAMPLES

### Example 1

### Testing the Efficacy of Conjugate MW001 in a Model of Multiple Sclerosis Induction of Experimental Autoimmune Encephalomyelitis (EAE)

Disease was induced in female C57B1 mice (6-8 weeks old; Harlan). Myelin Oligodendrocyte Glycoprotein (MOG) 35-55 was dissolved in saline (3 mg/ml) and emulsified (1:1) with Complete Freund's Adjuvant (CFA) (gibco; containing 5 mg of killed mycobacterium tuberculosis (MT). 200 μl of the emulsion (containing 300 μl of MOG) were injected into the right flank of each mouse, wherein and each mouse received a 250 ng supplement of pertussis toxin (PT, List) by intraperitoneal injection. Another 250 ng of PT were injected into the right flank of each mouse 24 hours later. The mice were observed daily from the 9th day post-EAE and the clinical signs were scored as described in Table 1 below:

TABLE I
evaluation of the clinical EAE signs

| Score | Signs | Description |
|---|---|---|
| 0 | Normal behavior | No neurological signs |
| 1 | Distal limp tail | The distal part of the tail |
| | | is limp and droops |
| 1.5 | Complete limp | The whole tail is loose |
| | tail | and droops |
| 2 | Righting reflex | The animal has difficulties |
| | | returning to its feet when |
| | | it is laid on its back |
| 3 | Ataxia | Wobbly walk-when the mouse |
| | | walks, its hind legs |
| | | are unsteady |
| 4 | Early paralysis | The mouse has difficulties |
| | | standing on its hind legs but still |
| | | has remnants of movement |
| 5 | Full paralysis | The mouse can't move its legs |
| | | at all, it looks thinner and |
| | | emaciated and suffers from |
| | | incontinence |
| 6 | Moribund/Death | Must be sacrificed |

### Treatment Paradigm

The MW-001 conjugate was dissolved in ethanol (60 mg/ml) and administered by intraperitoneal injection, at a dose of 50 mg/kg (10% stock in saline) twice a day, once at 9 am and once at 14 pm. Each dose was administered in a volume of 0.2ml/mouse (˜20 g). One group of animals was treated starting at day 0 (relating to the first day on which the induction of the disease started) and the other starting at day 10, when clinical signs first appeared. The experiment was continued for one month.

### Interpretation of Results

Calculation of the incidence of disease: The number of sick animals in each group was summed and percentage of sick animals in each group was calculated.

Calculation of the mean maximal score (MMS): The maximal scores of each of the 10 mice in the group was summed and the mean maximal score of each group was calculated as follows:Σmaximal score of each mouse/number of mice in the group.

Calculation of the group mean score (GMS):The scores of each of the 10 mice in the group was summed and the mean score per day calculated. The group mean score was calculated as follows: Σtotal score of each mouse per day/number of mice in the group.

Mean maximal score (MMS): defined as the disease severity; scored on a scale of 0-5 with graduations of 0.5 for intermediate clinical signs. The score is described in table-1

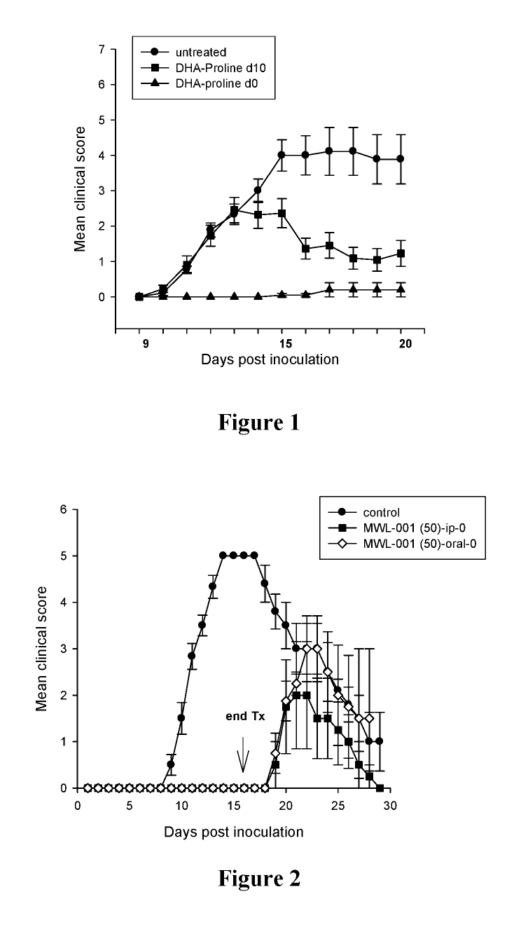

### Results

| | | | | | The results of |
|---|---|---|---|---|---|
| | | | | | the above |
| | | | | | study are |
| | | | | | presented in |
| | | | | | Figure 1 and |
| | | | | | in Table 2 |
| | | | | | below, |
| | | | | | wherein the |
| | | | | | animals were |
| | | | | | monitored |
| | | | | | once a day |
| | | | | | from day 0 to |
| | Incidence | | | | day 20 of the |
| | (#dead, | | Mean disease | | experiment. |
| | including | Mean maximal | duration | Mean day of | Mean score |
| Group | sacrificed) | score | (days) | onset | (whole group) |
| Untreated | 9/9 (2)* | 4.44 ± 0.19 | 10.33 ± 0.25 | 10.66 ± 0.06 | 2.67 ± 0.13 |
| MW-001 | 11/11 | 2.72 ± 0.14 | 8.90 ± 0.2 | 11 ± 0.09 | 1.34 ± 0.08 |
| (treatment | | | | | |
| started on | | | | | |
| day 10) | | | | | |
| MW-001 | **1/10 | 0.075 ± 0.02 | 0.6 ± 0.19 | 15 | 0.2 ± 0.06 |
| (treatment | | | | | |
| started on | | | | | |
| day 0) | | | | | |

*9/9(2)-Nine animals developed MS out of a group of 9 animals, wherein two animals died during the experiment.
**1/10-one animal developed MS out of a group of 10 animals.

As presented in FIG. 1 and in Table II above, the treated groups present results showing the effectiveness of the administered conjugate when compared to the untreated group, in all parameters tested. Further, a comparison between the two treated groups shows that treatment starting on day 0 is more effective than treatment starting on day 10. Thus, the results presented show that the MW-001 conjugate is significantly and surprisingly highly effective in preventing and ameliorating EAE in mice, which teaches that, surprisingly, the same conjugate would be effective in treating and/or preventing multiple sclerosis.

### Example 2

### Testing the Efficacy of Oral MWL-001 in a Model of Brain Inflammation (MS) Methods

Induction of EAE: Disease was induced in female C57Bl mice (6-8 weeks old; Harlan). Myelin Oligodendrocyte Glycoprotein (MOG 35-55) (Hadassah ein karem) was dissolved in saline (3 mg/ml) and emulsified (1:1) with Complete Freund's Adjuvant (CFA) (gibco; containing 5 mg of killed MT). 200 microliter of emulsion (containing 300 mg of MOG) was injected into the right flank, and each mouse received a supplement of 250 ng of pertussis toxin (List) i.p. Another 250 ng of pertussis toxin (PT) were injected 24 hours later. Mice were observed daily from the 9th day post-EAE and the clinical signs were scored as described in Table III below.

TABLE III
Evaluation of the EAE clinical signs.

| Score | Signs | Description |
|---|---|---|
| 0 | Normal behavior | No neurological signs. |
| 1 | Distal limp tail | The distal part of the tail |
| | | is limp and drops. |
| 1.5 | Complete limp tail | The whole tail is loose |
| | | and drops. |
| 2 | righting reflex | Animal has difficulties to |
| | | return on his feet when it is |
| | | laid on his back |
| 3 | Ataxia | wobbly walk-when the mouse |
| | | walks the hind legs are |
| | | unsteady |
| 4 | early paralysis | The mouse has difficulties |
| | | standing on its hind legs but |
| | | still has remnants of movement. |
| 5 | Full paralysis | The mouse can't move its legs at |
| | | all, it looks thinner and |
| | | emaciated. Incontinence |
| 6 | Moribund/Death | Must be sacrificed |

### Treatment Paradigm:

MWL-001 was dissolved in ethanol (60 mg/ml), wherein in one group MWL-001 was administered ip and in a second group MWL-001 was administered orally by gavage at a dose of 50 mg/kg (10% stock in saline). The MWL-001 was administered once a day (from day 0) at 9 am. Treatment ended on day 15 post inoculation. Each dose was administered in a volume of 0.2 ml/mouse

A third group of animals was treated ip with a dose of 25 mg/kg starting on day 7 and ending on day 15

A fourth group of animals was treated orally with methylprednisolone (MP, 10 mg/kg) starting on day 7 and ending on day 15.

A fifth group of animals was treated with MP (10 mg/kg) and MWL-001(25 mg/kg) together starting on day 7 and ending on day 15.

The number of animals in each of the above five groups was between 9 and 11.

### Interpretation of Results:

Calculation of the incidence of disease: The number of sick animals in each group was summed and percentage of sick animals in each group calculated.

Calculation of the mean maximal score (MMS): The maximal scores of each of the 10 mice in the group were summed and the mean maximal score of each group was calculated as follows:Σ maximal score of each mouse / number of mice in the group.

Calculation of the group mean score (GMS): The scores of each of the 10 mice in the group was summed and the mean score per day calculated. The group mean score was calculated as follows: Σ total score of each mouse per day/number of mice in the group.

### Results:

A dose of 50 mg/kg given once a day either orally or ip was found effective in inhibiting the clinical signs of the disease. A few days after cessation of the drug, mild signs of the disease appeared for a short period of time, as presented in FIG. 2.

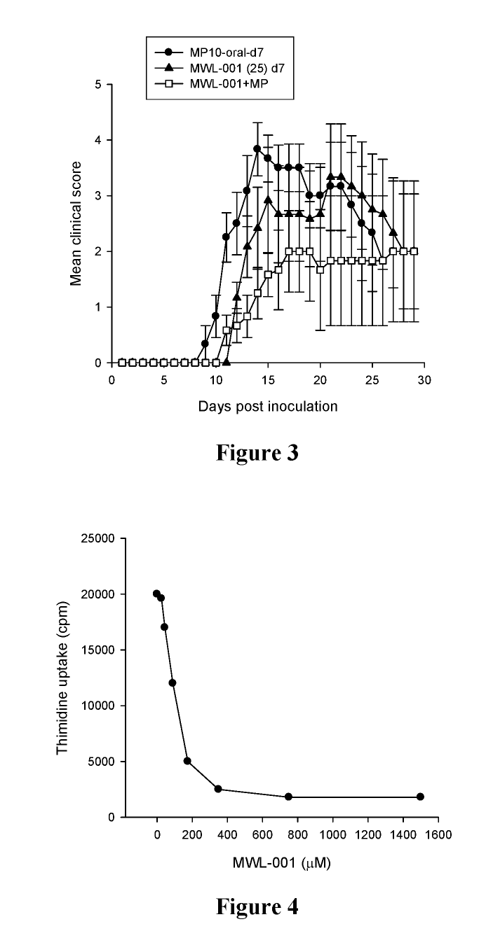

MWL-001 at a dose of 25 mg/kg given seven days post inoculation inhibited the severity of disease (3 vs 5 in the control group), but did not inhibit their appearance on day 10-11 post inoculation, just as the control untreated group. A dose of MP (10 mg/kg) was not effective in inhibiting the disease but a combination of both drugs more effective as compared to each one separately, as presented in FIG. 3.

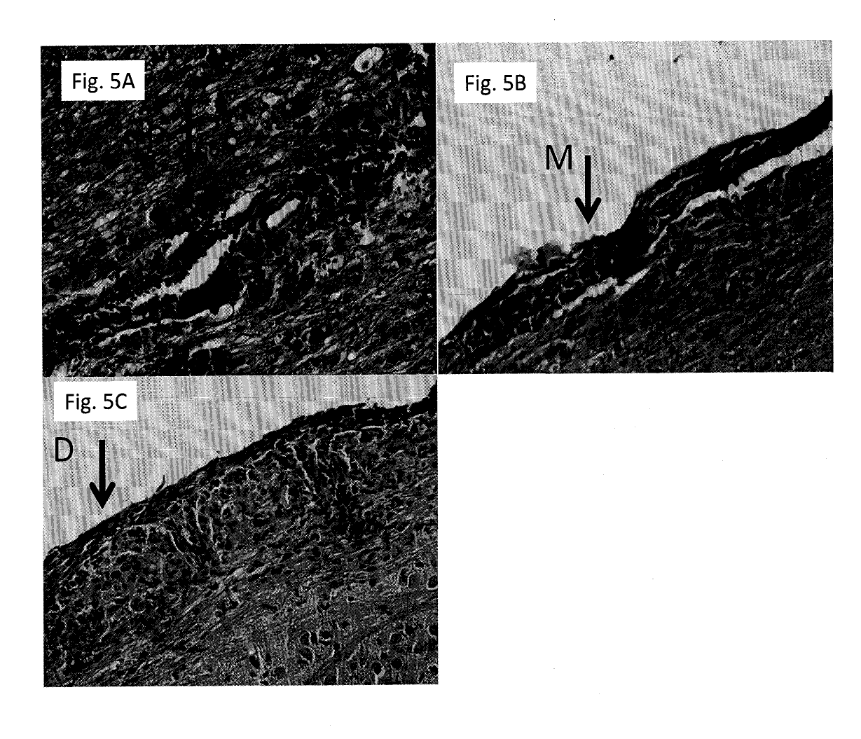

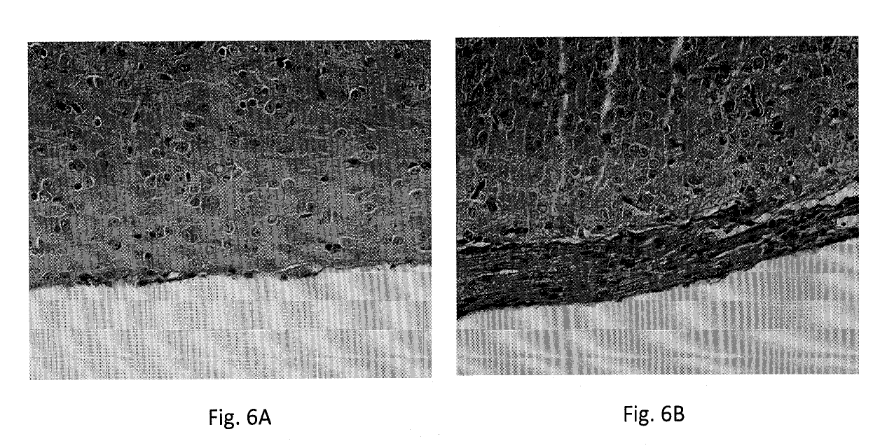

Histopathological photographs are presented as FIGS. 5, 6 and 7). FIG. 5 A-C shows massive mononuclear cells infiltrates in the spinal cord of control EAE untreated animal. In contrast, as can be seen, very few meningeal infiltrates can be seen in the animal treated with MWL-001 administered i.p. on Day 0 as compared to untreated (FIG. 6 A and B), see details in Example 2.

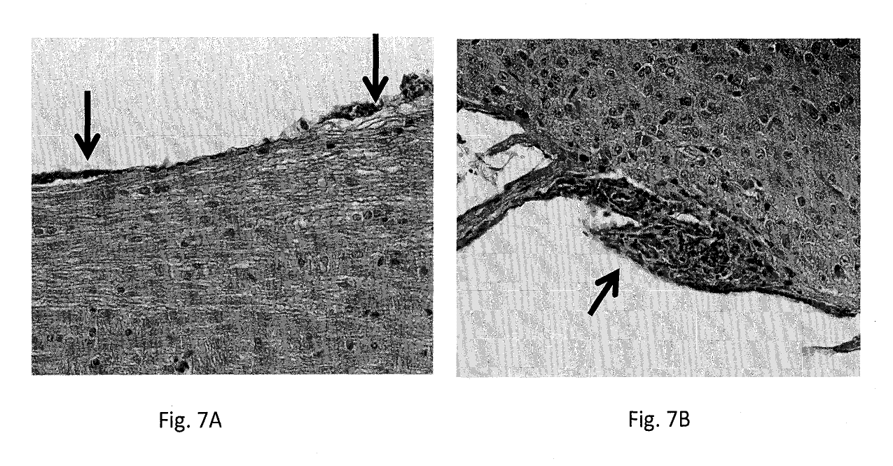

Further, in DHA-proline (MWL-001) treated EAE animal (the i.p. treatment started at day 10) very few meningeal infiltrates can be observed. Further, there is no parenchymal or diffused infiltrates (FIG. 7 A and B).

### In Vitro Effect of MWL-001 on the Activation of Human Lymphocytes

This experiment was conducted in order to elucidate the mechanism of action of MWL-001 in multiple sclerosis MS). It is believed that activated lymphocytes have a crucial role in developing MS.

Mononuclear cells were separated from whole blood on a gradient of phicoll-hypaque Cells (10⁵ per well) and were incubated in 96 well plate (nunclon) in enriched medium (RPMI containing antibiotics, glutamine, sodium pyruvate, mercaptoethanol and 10% FCS) and incubated in the absence or presence of phytohemagglutinin (PHA), which is a non specific mitogen of T cells. MWL-001 was dissolved in complete medium of the cells and added immediately to the cells. Control cells were added with the vehicle of MWL-001.

Cells were incubated for 4 days in a humidified incubator at a temperature of 37° C. ³H-thymidine was added on day 3 and then cells were harvested and counted in a beta scintillation counter. The results are presented in FIG. 4.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. Method for treating multiple sclerosis (MS) comprising administering a conjugate of hydroxyproline and docosahexaenoic acid (DHA) according to formula MW-001:

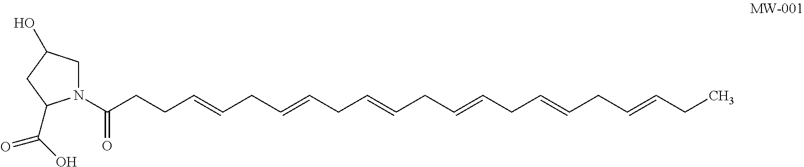 [SMILES: CC/C=C/C/C=C/C/C=C/C/C=C/C/C=C/C/C=C/CCC(=O)N1CC(O)CC1C(=O)O]

2. Method for preventing the outburst of multiple sclerosis (MS) comprising administering a conjugate of hydroxyproline and docosahexaenoic acid (DHA) according to formula MW-001:

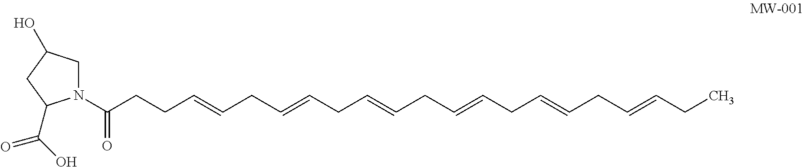 [SMILES: CC/C=C/C/C=C/C/C=C/C/C=C/C/C=C/C/C=C/CCC(=O)N1CC(O)CC1C(=O)O]

3. Method for preventing the continual chronic deterioration caused by multiple sclerosis (MS) comprising administering a conjugate of hydroxyproline and docosahexaenoic acid (DHA) according to formula MW-001:

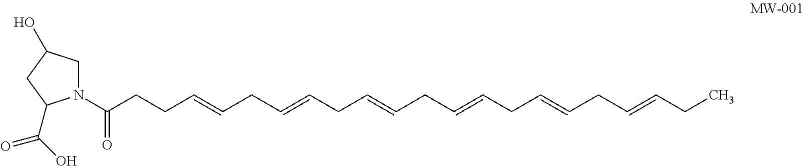 [SMILES: CC/C=C/C/C=C/C/C=C/C/C=C/C/C=C/C/C=C/CCC(=O)N1CC(O)CC1C(=O)O]

4. Method for treating symptoms of multiple sclerosis (MS) comprising administering a conjugate of hydroxyproline and docosahexaenoic acid (DHA) according to formula MW-001:

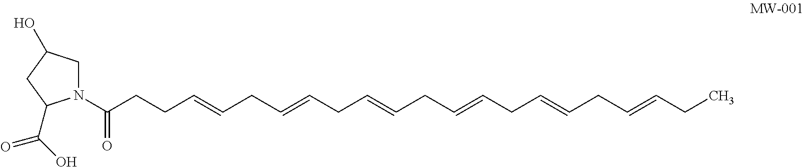 [SMILES: CC/C=C/C/C=C/C/C=C/C/C=C/C/C=C/C/C=C/CCC(=O)N1CC(O)CC1C(=O)O]

5. The method according to claim 1 comprising administering the conjugate orally, parenterally, by nasal administration, pulmonary administration or any combination thereof.

6. The method according to claims claim 1, wherein the conjugate is administered together with a pharmaceutical carrier.

7. The method according to claim 6, wherein the pharmaceutical carrier is a gas, liquid or solid.

8. The method according to claim 6, wherein the pharmaceutical carrier is saline solution, water, an emulsion, a dispersion or any combination thereof.

9. The method according to claim 1, wherein the conjugate is administered in a modified release form.

10. The method according to claim 1, wherein the conjugate is administered together with an additional active ingredient.

11. The method according to claim 10, wherein the additional active ingredient is selected from Interferon beta 1a, Interferon beta 1b, Glatiramer acetate, mitoxantrone, methyl-prednisolone, prednisone, prednisolone, dexamethasone, adreno-corticotrophic hormone (ACTH), corticotrophin, beta interferons, corticostaeorids, natalizumab (Tysabri®), fingolimod (Gilenya®), glatiramer acetate (Copaxone®), mitoxantrone teriflunomide (Aubagio®) or any appropriate combination thereof.

12. The method according to claim 1, wherein the conjugate is administered in an amount of between 1 mg/kg to 1000 mg/kg.

13. The method according to claim 1, wherein the conjugate is administered chronically, during flare ups, before the outbreak of the disease or any combination thereof.

14. The method according to claim 1, wherein the conjugate is administered once a day, twice a day, three times a day or at least once a day.

15. The method according to claim 1, wherein the conjugate is administered for 10 days, 20 days, 30 days, 60 days, 90 days, 120 days or chronically.
